(19) United States
(12) Patent Application Publication
(10) Pub. No.: US 2023/0263751 A1
(43) Pub. Date: Aug. 24, 2023

(54) Compositions and Methods of Use of beta-hydroxy-beta-methylbutyrate (HMB) and Chemotherapy Agents

(71) Applicant: Metabolic Technologies, LLC, Missoula, MT (US)

(72) Inventors: Karina Bettega Felipe, Parana (BR); Raquel Goreti Eckert Dreher, Parana (BR); Ronagela Curi Pedrosa, Santa Catarina (BR); Lisa Pitchford, Ames, IA (US)

(21) Appl. No.: 18/010,582

(22) PCT Filed: Jun. 15, 2021
(86) PCT No.: PCT/US21/37431

§ 371 Date: Dec. 15, 2022

Related U.S. Application Data
(60) Provisional application No. 63/038,989, filed on Jun. 15, 2020.

(51) Int. Cl. A61K 31/19; A61K 31/704; A61P 35/00; A61P 29/00; A61P 3/00

(52) U.S. Cl. CPC A61K 31/19; A61K 31/704; A61P 35/00; A61P 29/00; A61P 3/00

(57) ABSTRACT

The present invention provides methods of administering HMB to mammals undergoing chemotherapy treatment or receiving a chemotherapy agent to inhibit tumor growth, increase animal survival, protect against chemotherapy-induced weight loss, protect against chemotherapy-induced inflammation, and/or provide an anti-cachectic treatment.

This application claims the benefit of U.S. Provisional Patent Appln. No. 63/038,989 filed Jun. 15, 2020 and herein incorporates U.S. Provisional Patent Appln. No. 63/038,989 by reference.

### BACKGROUND OF THE INVENTION

### 1. Field

The present invention relates to compositions and methods of use of β-hydroxy-β-methylbutyrate (HMB) and chemotherapy agents to inhibit tumor growth, increase animal survival, protect against chemotherapy-induced weight loss, protect against chemotherapy-induced inflammation, and/or provide an anti-cachectic treatment.

### 2. Background

Chronic inflammation occurs in several types of cancer, and this process is linked to tumor progression. Neoplastic cells synthesizes cytokines and chemokines that attract macrophages and other inflammatory cells that make up the tumor microenvironment; these cells produce cytotoxic mediators (ROS, TNFα, interleukins and interferons) that contribute to tumor growth, metastasis and angiogenesis [1,2].

The inflammatory state associated with tumor growth also contributes to increased protein catabolism and the risk of developing cachexia [3]. The pathophysiological and biochemical mechanisms of cachexia are complex, involving factors that increase lipid and protein mobilization, chronic inflammation status as host response to tumor presence, as well as changes in energy metabolism [4,5]. In this context, the synthesis of pro-inflammatory cytokines, such as IL-1 and IL-6, contributes to the aggravation of cachexia, considering that they act directly on target tissues, promoting depletion of skeletal and adipose muscle tissue. In addition, they interact with the central nervous system, interfering with food intake and energy metabolism [6].

Additional research also suggests that chemotherapy can also contribute to the development of cachexia [7]. The doxorubicin (Doxo), as a first line chemotherapeutic agent, causes reduction of skeletal and cardiac muscles and was associated with activation of p53-p21-REDD1 (protein regulated in development and DNA damage response 1) pathway. The doxorubicin can also reduce protein synthesis and activating proteolysis and apoptosis signaling, and also suppressing the expression of genes associated with lipogenesis, PUFA (polyunsaturated fatty acids) biosynthesis and fatty acid uptake [8,9]. Considering these findings and the results of a clinical trial with women with breast cancer treated with doxorubicin and cyclophosphamide, where there was an increase in the diagnosis of malnutrition comparing the beginning and the end of chemotherapy (15 and 38%, respectively) [10], it is believed that Doxo can induce cachexia.

Cachexia is related to worse prognosis, longer hospital stays and higher patient mortality [11,12]. In this sense, HMB, a leucine metabolite produced in vivo in the cytosol by the α-ketoisocaproic acid (KIC) pathway, has been the subject of several investigations in malnutrition because it stimulates protein synthesis through the mTOR/p70S6k pathway [13]. It has also been found that HMB is capable of promoting muscle mass gain in athletes, as well as exerting anticatabolic effect in bedridden elderly [14]. The only product of leucine metabolism is ketoisocaproate (KIC). A minor product of KIC metabolism is β-hydroxy-β-methylbutyrate (HMB). HMB has been found to be useful within the context of a variety of applications. Specifically, in U.S. Pat. No. 5,360,613 (Nissen), HMB is described as useful for reducing blood levels of total cholesterol and low-density lipoprotein cholesterol. In U.S. Pat. No. 5,348,979 (Nissen et al.), HMB is described as useful for promoting nitrogen retention in humans. U.S. Pat. No. 5,028,440 (Nissen) discusses the usefulness of HMB to increase lean tissue development in animals. Also, in U.S. Pat. No. 4,992,470 (Nissen), HMB is described as effective in enhancing the immune response of mammals. U.S. Pat. No. 6,031,000 (Nissen et al.) describes use of HMB and at least one amino acid to treat disease-associated wasting.

Some investigators have described distinct effects of HMB on alteration in tumor biology. Smith et al [15] showed that in addition to having a dose-dependent effect on weight loss, HMB also led to significant reduction in tumor growth rate. Caperuto et al [16] showed enhanced survival time in rats implanted with subcutaneous tumors and improved survival by 42% when the tumors were injected into the peritoneal cavity. HMB also reduced tumor growth rate [17], tumor weights, and tumor cell proliferation rates in rats by enhancing apoptosis [18].

In recent years, the anti-inflammatory effect of HMB has also been discussed in animal models subjected to radiation and patients with head and neck cancer undergoing radiotherapy [19,20].

The present invention provides methods of administering HMB to mammals undergoing chemotherapy treatment to inhibit tumor growth, increase animal survival, protect against chemotherapy-induced weight loss, protect against chemotherapy-induced inflammation, and/or provide an anti-cachectic treatment.

### SUMMARY OF THE INVENTION

One object of the present invention is to provide a combination treatment of a chemotherapy agent and HMB to inhibit tumor growth.

Another object of the present invention is to provide a combination of a chemotherapy agent and HMB to increase animal survival.

An additional object of the present invention is to provide a combination treatment of a chemotherapy agent and HMB to protect against chemotherapy-induced weight loss.

A further object of the present invention is to protect against chemotherapy-induced inflammation.

Another object of the present invention is to provide HMB to a mammal undergoing chemotherapy treatment for anti-cachectic activity.

The present invention intends to overcome the difficulties encountered heretofore. To that end, a composition comprising HMB administered in association with a chemotherapy regimen is provided. The composition is administered to a subject in need thereof. All methods compromise administering to the animal HMB in association with chemotherapy. The subjects included in this invention include humans and non-human mammals. The composition is consumed by a subject in need thereof.

### BRIEF DESCRIPTION OF THE DRAWINGS

FIG. 1(A) depict inhibition of tumor growth (%).

FIG. 1 (B) depicts survival of mice.

FIG. 2 depicts type of cell death induced in EAC cells and expression of proteins related to cell death.

FIG. 3 depicts body composition assessment of mice.

FIG. 4 depicts muscle and systemic inflammatory parameters mice.

### DETAILED DESCRIPTION OF THE INVENTION

It has been surprisingly and unexpectedly discovered that administration of HMB in conjunction with a chemotherapy protocol results in inhibited tumor growth and increased animal survival. HMB is protective against chemotherapy-induced weight loss and inflammation. The protective effects do not interfere with anti-tumor action of the chemotherapy agent.

HMB

β-hydroxy-β-methylbutyric acid, or β-hydroxy-isovaleric acid, can be represented in its free acid form as (CH₃)₂(OH)CCH₂COOH. The term “HMB” refers to the compound having the foregoing chemical formula, in both its free acid and salt forms, and derivatives thereof. While any form of HMB can be used within the context of the present invention, preferably HMB is selected from the group comprising a free acid, a salt, an ester, and a lactone. HMB esters include methyl and ethyl esters. HMB lactones include isovalaryl lactone. HMB salts include sodium salt, potassium salt, chromium salt, calcium salt, magnesium salt, alkali metal salts, and earth metal salts.

Methods for producing HMB and its derivatives are well-known in the art. For example, HMB can be synthesized by oxidation of diacetone alcohol. One suitable procedure is described by Coffman et al., J. Am. Chem. Soc. 80: 2882-2887 (1958). As described therein, HMB is synthesized by an alkaline sodium hypochlorite oxidation of diacetone alcohol. The product is recovered in free acid form, which can be converted to a salt. For example, HMB can be prepared as its calcium salt by a procedure similar to that of Coffman et al. (1958) in which the free acid of HMB is neutralized with calcium hydroxide and recovered by crystallization from an aqueous ethanol solution. The calcium salt of HMB is commercially available from Metabolic Technologies, Ames, Iowa.

Calcium β-hydroxy-β-methylbutyrate (HMB) Supplementation

More than 2 decades ago, the calcium salt of HMB was developed as a nutritional supplement for humans. Studies have shown that 38 mg of CaHMB per kg of body weight appears to be an efficacious dosage for an average person.

The molecular mechanisms by which HMB decreases protein breakdown and increases protein synthesis have been reported. Eley et al conducted in vitro studies which have shown that HMB stimulates protein synthesis through mTOR phosphorylation. Other studies have shown HMB decreases proteolysis through attenuation of the induction of the ubiquitin-proteosome proteolytic pathway when muscle protein catabolism is stimulated by proteolysis inducing factor (PIF), lipopolysaccharide (LPS), and angiotensin II. Still other studies have demonstrated that HMB also attenuates the activation of caspases-3 and -8 proteases.

### HMB Free Acid Form

In most instances, the HMB utilized in clinical studies and marketed as an ergogenic aid has been in the calcium salt form. Recent advances have allowed the HMB to be manufactured in a free acid form for use as a nutritional supplement. A free acid form of HMB was developed, which was shown to be more rapidly absorbed than CaHMB, resulting in quicker and higher peak serum HMB levels and improved serum clearance to the tissues.

HMB free acid may therefore be a more efficacious method of administering HMB than the calcium salt form, particularly when administered directly preceding intense exercise. One of ordinary skill in the art, however, will recognize that this current invention encompasses HMB in any form.

HMB in any form may be incorporated into the delivery and/or administration form in a fashion so as to result in a typical dosage range of about 0.5 grams HMB to about 30 grams HMB.

Any suitable dose of HMB can be used within the context of the present invention. Methods of calculating proper doses are well known in the art. Methods of calculating proper doses are well known in the art. The dosage amount of HMB can be expressed in terms of corresponding mole amount of Ca-HMB. The dosage range within which HMB may be administered orally or intravenously is within the range from 0.01 to 0.2 grams HMB (Ca-HMB) per kilogram of body weight per 24 hours. For adults, assuming body weights of from about 100 to 200 lbs., the dosage amount orally or intravenously of HMB (Ca-HMB basis) can range from 0.5 to 30 grams per subject per 24 hours.

It will be understood by one of ordinary skill in the art that HMB and the chemotherapy agent(s) do not have to be administered in the same composition or at the same time to perform the claimed methods.

The term administering or administration includes providing a composition to a mammal, consuming the composition and combinations thereof.

### Experimental Examples

The following examples will illustrate the invention in further detail. It will be readily understood that the composition of the present invention, as generally described and illustrated in the Examples herein, could be synthesized in a variety of formulations and dosage forms. Thus, the following more detailed description of the presently preferred embodiments of the methods, formulations and compositions of the present invention are not intended to limit the scope of the invention, as claimed, but it is merely representative of the presently preferred embodiments of the invention.

### Materials and Methods

### Animals and Experimental Designs

Female (18 to 23 g weight, 60 days) Balb/c mice (Mus musculus) obtained from controlled reproduction carried out in the sectoral bioterium of the Federal University of Santa Catarina (Brazil), were kept in plastic cages under controlled environmental conditions (12 h light-dark cycle, 25±2° C., relative humidity 60%), receiving commercial feed and water ad libitum.

The mice were divided into 5 groups (n=6): Normal (healthy animals), Control (saline), Doxo (1 mg/kg/day), HMB (617.3 mg calcium HMB/kg/day) and Doxo+HMB (1 mg/kg/day and 617.3 mg/kg/day, respectively). The animals belonging to the Control, Doxo, HMB and Doxo+HMB groups were inoculated with EAC cells (200 μL, 5×10⁶ cells) by intraperitoneal injection and treatment of animals was started after 96 hours. The dose of HMB was defined based on the recommendation for adult men of 70 kg, that is, 3 g/day (supplementary form) [21].

Animal studies were conducted with isogenic Balb/c mice housed and received treatments accordingly with legal requirements (NIH publication #80-23, revised in 1985) and local Ethics Committee for Animal Use (approved protocol CEUA/UFSC PPOO784).

### Evaluation of Antitumor Effect

### Tumor Growth Inhibition and Survival Assessment

The moment before the tumor was inoculated, the abdominal circumference was measured. The treatments were administered intraperitoneally for 9 consecutive days. Twenty-four hours after the last treatment, all mice were weighed and their abdominal circumferences measured again. The inhibition (%) of tumour growth was calculated as follow [22]: [(average in waist circumference of the treated group×100)/average in waist circumference of the control group]−100.

In addition, after euthanasia, all ascitic fluid was collected to measure volume and weight. Finally, Balb/c mice (n=12) were chosen at random and kept alive to assess the effects of HMB, Doxo and Doxo+HMB on survival time according to Kaplan and Meier (1958) [23]; survival assessments were discontinued after 30 days.

### Assessment of Death Type

Harvested tumor cells (5×10⁶) were stained with 1 μL of a solution (1:1) of propidium iodide (100 μg/mL) and acridine orange (100 μg/mL). The samples were read by fluorescence microscopy on the green (excitation at 460 nm and emission at 520 nm) and red (excitation at 492 nm at emission at 620 nm) filters, and the results were expressed as percentage of viable (green), apoptotic (orange) and necrotic (red) cells [24].

### Western Blotting

Apoptotic markers were evaluated by Western blotting. EAC cells were washed with PBS and lysed in RIPA buffer supplemented with 1% protease and 3% phosphatase inhibitors. The proteins were further denatured in Laemmli buffer and equivalent amounts were subjected to SDS-PAGE electrophoresis, followed by electroblotting on PVDF membranes. The membranes were blocked and then incubated with the primary monoclonal antibodies: p53 (Santa Cruz Biotechnology, DO-1; sc-126), Bax (Santa Cruz Biotechnology, B-9; sc-7480) and BcL-xl (Santa Cruz Biotechnology, H-5; sc-8392), and subsequently incubated with peroxidase conjugated secondary antibodies (Dako and Chemicon). Immunodetection was performed using a chemiluminescence determination kit, and β-actin was used as a loading control. Images were obtained with the ChemiDoc MP (Bio Rad) system [25].

### Cachexia Assessment

To determine average daily food intake, the weight of the feed consumed was divided by the number of animals in each cage [26]. To evaluate the percentage of weight loss, the “initial weight” (before inoculation of the EAC), “final weight” (after 9 days of treatment) and “carcass weight” (final weight−weight of ascitic liquid) were considered according to standardized equation [27].

The soleus and gastrocnemius muscles were quickly removed and weighed on an analytical balance (wet mass). Soleus samples were used to determine the dry weight after 3 days at 60° C., and the gastrocnemius was kept at −80° C. for the determination of cytokines. The wet mass of the gastrocnemius and soleus muscle (mg) was normalized by the length of the tibia (mm) of the mice [28]. Subcutaneous fat deposits, mesentery and brown adipose tissue were also removed and immediately weighed.

### Evaluation of the Inflammatory Profile

The COX-2 expression in EAC cells was assessed by Western blotting as described in item 2.2.3. Antibodies to COX-2 were purchased from Cell Signaling (#4842)

For the determination of cytokines, the gastrocnemius muscle was homogenized in a RIPA buffer supplemented with 1% protease and 3% phosphatase inhibitors, in the proportion 1 mg tissue:10 μl buffer, followed by centrifugation of the homogenate at 12,000 g and 4° C., for 10 minutes. An aliquot of the supernatant was used to determine the cytokines IL-1β and IL-6 by ELISA immunoassay using DuoSet® Kit (R&D System, USA), according to the manufacturer recommendations.

The determination of C-reactive protein (CRP) in serum was performed by the Ultra Turbiquest Plus® CRP kit, according to the manufacturer instructions.

### Statistical Analysis

Statistical analysis was performed using Prism 5 for Windows, version 5.00, using one-way analysis of variance (ANOVA), with Tukey post hoc test, assuming a minimal significance level of p<0.05.

### Results

Characterization of the Antitumor Effect of Doxorubicin Associated with HMB

All treatments promoted a significant reduction in the variation of body weight (difference between the weight before the tumor inoculation and on the day of euthanasia) and the volume of ascitic fluid. The treatment with Doxo reduced the weight variation by 39% and the volume of ascitic liquid by 54% in relation to the Control group. The treatment with Doxo+HMB reduced the same parameters by 43% and 37%, respectively (Table 1). These results are highlighting the antitumor potential of the association Doxo+HMB in EAC. There was no statistical difference between Doxo and Doxo+HMB groups.

TABLE 1
Morphological evaluations of healthy (Normal) Balb/c mice
and mice with EAC, treated with saline (Control), Doxo
(1 mg/kg/day), HMB (617.3 mg/kg/day), and Doxo + HMB
(1 mg/kg/day and 617.3 mg/kg/day, respectively) for 9 days.

| | Body weight | Ascitic fluid |
|---|---|---|
| Treatment | variation (g) | volume (mL) |
| Normal | 0.2 ± 0.2^{α}***^{, β}** | — |
| Control | 14.4 ± 2.5 | 16.5 ± 2.4 |
| Doxo | 8.8 ± 1.6^{α}* | 7.6 ± 1.3^{α}*** |
| HMB | 8.8 ± 1.4^{α}* | 10.3 ± 1.3^{α}** |
| Doxo + HMB | 8.2 ± 2.6^{α}* | 10.4 ± 1.4^{α}** |

Results are expressed as mean ± standard deviation,
n = 6,
*p < 0.05,
**p < 0.01,
***p < 0.001 compared to the negative control (^{α}) and Doxo (^{β}).

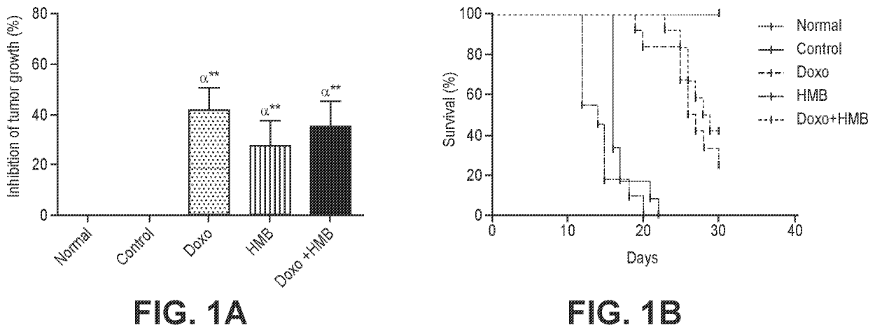

The treatments with Doxo and Doxo+HMB were able to inhibit tumor growth by 42% and 39%, respectively, in relation to the Control group, however, there was no statistical difference between them, demonstrating that HMB does not interfere with the antitumor effect of Doxo (FIG. 1A). FIG. 1 shows (A) Inhibition of tumor growth (%) and (B) survival of healthy Balb/c mice and mice with EAC, treated with saline (Control), Doxo (1 mg/kg/day), HMB (617.3 mg/kg/day), and Doxo+HMB (1 mg/kg/day and 617.3 mg/kg/day, respectively) for 9 days. Results are expressed as mean±standard deviation, n=6 (A) and n=12 (B), *p<0.05, **p<0.01, ***p<0.001 compared to the negative control (u).

The animal mean survival time was 16 days (Control), 13.5 days (HMB) and 26 days (Doxo and Doxo+HMB) (FIG. 1B). In this experimental model treatments with Doxo and Doxo+HMB were able to increase the animal survival in relation to the Control group, however, at the end of the observation period, there were 3 surviving animals in Doxo group and 5 animals in Doxo+HMB.

### 3.2 Association Doxo+HMB Induces Cell Death by Apoptosis

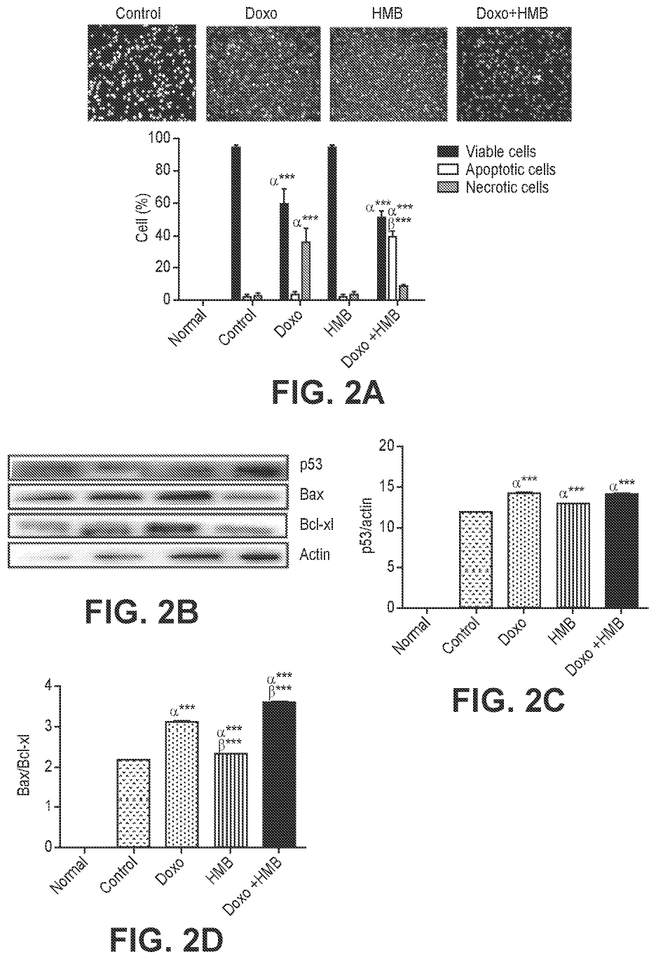

FIG. 2 shows the type of cell death induced in EAC cells and expression of proteins related to cell death. (A) Type of cell death induced, (B) Expression of p53, Bax and Bcl-xl, (C) Expression of p53/actin, and (D) Bax/Bcl-xl ratio. Results are expressed as mean±standard deviation, n=6, *p<0.05, **p<0.01, ***p<0.001 compared to Control (α) and Doxo (β).

Doxo significantly reduced the number of viable cells (60%), inducing necrosis (36%) and apoptotic cell death (4%) (FIG. 2A). On the other hand, treatment with Doxo+HMB was able to similarly reduce the number of viable cells (52%) by increasing the number of apoptotic cells (39%) without inducing significant necrosis (9%). Both Doxo and Doxo+HMB significantly increased the Bax/Bcl-xL ratio in relation to the Control (Doxo 38% and Doxo+HMB 60%), with significant difference between Doxo+HMB and Doxo (FIGS. 2B and 2D). All treatments similarly increased the expression of p53 in relation to the Control group (Doxo 20%, HMB 10% and Doxo+HMB 19%; FIGS. 2B and 2C). It is important to note that cell death by necrosis is associated with the inflammatory process while apoptosis does not induce inflammation.

3.3 Co-Treatment with Doxorubicin and HMB Modulates Tumor Cachexia

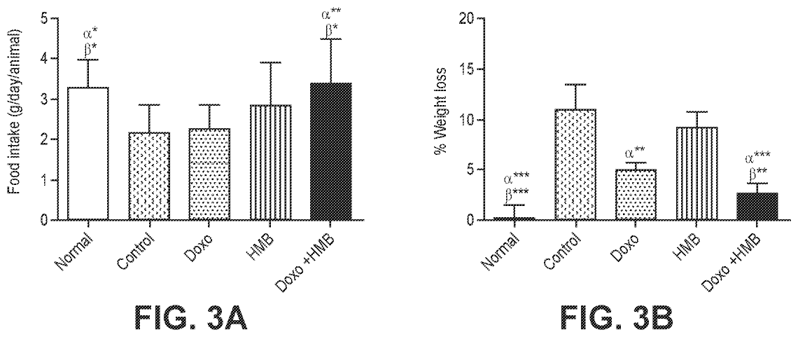

FIG. 3 shows body composition assessment of healthy Balb/c mice and mice with EAC treated with saline (Control), Doxo (1 mg/kg/day), HMB (617.3 mg/kg/day) and Doxo+HMB (1 mg/kg/day and 617.3 mg/kg/day, respectively) for 9 days. (A) Food intake (g/day) and (B) Weight loss (%). Results were expressed as mean±standard deviation, n=6 (A and B) and n=12 (C), *p<0.05, **p<0.01, ***p<0.001 compared to Control (α) and Doxo (β).

The average daily food intake among the groups (FIG. 3A) was as follows: 3.30±0.60 g/day for the Normal group; 2.17±0.69 g/day for the Control group; 2.22±0.63 g/day for the Doxo group; 2.81±1.05 g/day for the HMB group; and 3.39±1.06 g/day for the Doxo+HMB group. The food intake of the groups treated with Doxo and HMB alone were not statistically different from the Control group; however, co-treatment with Doxo+HMB promoted an increase in food intake in relation to the Control and Doxo groups.

Inoculation of EAC cells caused a significant loss of body weight (11%). Treatment with Doxo and Doxo+HMB attenuated the weight loss in relation to controls (54% and 75%, respectively). Furthermore, we observed a statistical difference between Doxo and Doxo+HMB groups, showing that Doxo+HMB was more effective than Doxo alone in preserving body mass (FIG. 3B).

There were no statistical difference between groups for wet or dry soleus muscle mass (Table 2); however, inoculation with EAC cells reduced the wet mass of the gastrocnemius muscle by 35% compared to the Normal group. Though neither Doxo or HMB alone affected gastrocnemius mass, co-treatment with Doxo+HMB increased the mass by 47% and 26% in relation to the control group and Doxo alone, respectively (Table 2).

TABLE 2
Evaluation of body compartments (wet and dry weight of the muscles gastrocnemius and soleus, weight of subcutaneous
and mesenteric fat and brown adipose tissue) of Balb/c mice with EAC treated with Doxo (1 mg/kg/day), HMB
(617.3 mg/kg/day) and Doxo + HMB (1 mg/kg/day and 617.3 mg/kg/day, respectively) for 9 days.

| | Normal | Control | Doxo | HMB | Doxo + HMB |
|---|---|---|---|---|---|
| Wet weight | 15.5 ± 1.4^{α}***^{, β}* | 10.1 ± 1.3 | 11.7 ± 0.2 | 8.9 ± 0.5 | 14.8 ± 1.4^{α}**^{, β}* |
| gastrocnemius muscle | | | | | |
| (mg/mm tibia length) | | | | | |
| Wet weight soleus | 0.7 ± 0.2 | 0.7 ± 0.03 | 0.7 ± 0.15 | 0.8 ± 0.2 | 0.7 ± 0.06 |
| muscle | | | | | |
| (mg/mm tibia length) | | | | | |
| Dry weight soleus | 0.2 ± 0.02 | 0.1 ± 0.02 | 0.1 ± 0.01 | 0.1 ± 0.04 | 0.1 ± 0.04 |
| muscle | | | | | |
| (mg/mm tibia length) | | | | | |
| Brown adipose tissue | 6.4 ± 3.9 | 3.5 ± 0.6 | 3.6 ± 1.7 | 3.2 ± 0.3 | 5.5 ± 0.3 |
| mass | | | | | |
| (mg/cm mouse length) | | | | | |
| Subcutaneous fat mass | 11.1 ± 3.4^{α}***^{, β}** | 0.6 ± 0.3 | 2.2 ± 0.3 | 1.9 ± 1.1 | 8.3 ± 2.1^{α}**^{, β}* |
| (mg/cm mouse length) | | | | | |
| Mesenteric fat mass | 11.9 ± 2.3^{α}***^{, β}*** | 4.0 ± 0.4 | 2.5 ± 0.6 | 3.0 ± 0.8 | 6.5 ± 1.7^{β}* |
| (mg/cm mouse length) | | | | | |

Results are expressed as mean ± standard deviation,
n = 6,
*p < 0.05,
**p < 0.01,
***p < 0.001 compared to the negative control (^{α}) and Doxo (^{β}).

In relation to healthy animals, inoculation with EAC promoted depletion of subcutaneous and mesenteric fat deposits by 94% and 66%, respectively. However, Doxo+HMB increased the subcutaneous fat deposits in relation to the Control and Doxo groups, protecting against the fat depletion induced by EAC and chemotherapy (Table 2). Doxo+HMB also promoted an increase in mesenteric fat compared to the Doxo group. However, treatment with the Doxo or HMB alone was not effective at maintaining subcutaneous and mesenteric fat compared to controls.

### 3.4 HMB Modulates Inflammatory Pathways in Both EAC and Skeletal Muscle

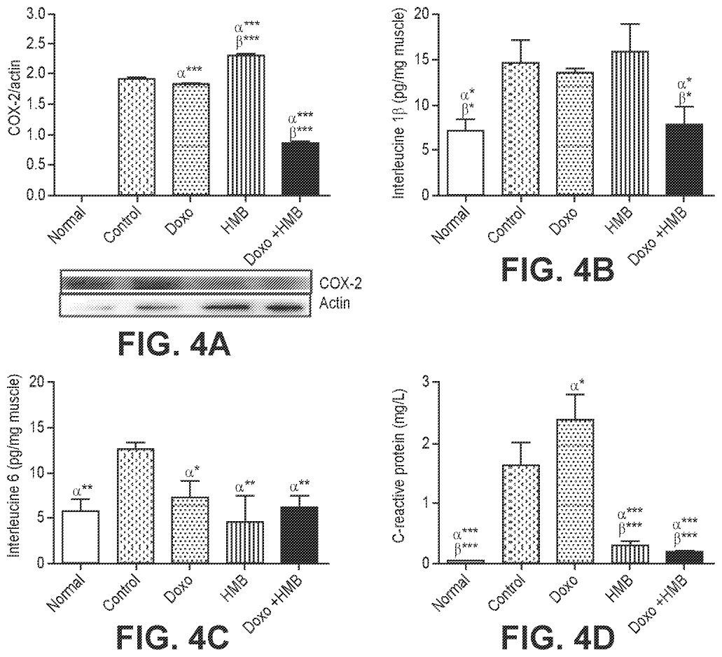

FIG. 4 shows muscle and systemic inflammatory parameters in healthy Balb/c mice and mice with EAC treated with saline (Control), Doxo (1 mg/kg/day), HMB (617.3 mg/kg/day) and Doxo+HMB (1 mg/kg/day and 617.3 mg/kg/day, respectively) for 9 days. (A) Expression of COX-2/actin in EAC cells, (B) Expression of IL-1β and (C) IL-6 in the gastrocnemius, and (D) Serum C-reactive protein levels (mg/L). Results are expressed as mean±standard deviation, n=6, *p<0.05, **p<0.01, ***p<0.001 compared to the negative control (α) and Doxo (β).

HMB and Doxo+HMB treatments reduced the EAC cell expression of COX-2 when compared to controls (4% and 55%, respectively). Doxo+HMB treatment also reduced the expression of COX-2 by 53% compared to Doxo alone (FIG. 4A).

In addition, it was observed that inoculation with EAC cells increased the expression of IL-1β in the gastrocnemius muscle by 103% compared to the Normal group. Doxo and Doxo+HMB treatments reduced the content of this cytokine in 6% and 47%, respectively, in relation to controls. Finally, the association Doxo+HMB reduced the expression of IL-1β by 43% compared to Doxo alone (FIG. 4B).

EAC also increased the expression of IL-6 in the gastrocnemius muscle by 116% compared to healthy animals, but this cytokine was reduced by 43%, 64% and 51%, in the Doxo, HMB and Doxo+HMB treatments, respectively, with no statistical difference between treatments (FIG. 4C).

Serum C-reactive protein levels were 0.07 mg/L (Normal), 1.63 mg/L (Control), 2.39 mg/L (Doxo), 0.31 mg/mL (HMB) and 0.2 mg/L (Doxo+HMB) (FIG. 4D). Statistical analyses showed that while Doxo further increased serum levels of C-reactive protein above those in the control group, both HMB alone and Doxo+HMB decreased this parameter in relation to both the control and Doxo groups.

### Discussion

The administration of Doxo+HMB inhibited tumor growth and increased animal survival compared to the Control, Doxo and HMB (alone) groups, HMB was protective against Doxo-induced weight loss and inflammation without interfering with the antitumor action of Doxo

Co-treatment with Doxo+HMB shifted the type of cell death induced by Doxo chemotherapy from necrosis to apoptosis, but did not alter the total amount of cell death, as the percentage of death induced by each treatment was similar (Doxo 36% and Doxo+HMB 39%). The association of Doxo+HMB increased Bax expression and decreased Bcl-xl expression in relation to the control and Doxo groups, modulating the Bax/Bcl-xl ratio and promoting intrinsic apoptosis. The benefit of such association is to induce apoptosis and to contribute to the reduction of inflammation in the tumor microenvironment.

COX-2 is overexpressed in several types of cancer, being related to a worse prognosis by favoring mutations, cell proliferation, induction of chemoresistance and reduction of apoptosis (related to the increased expression of anti-apoptotic proteins of the Bcl-2 family and reducing the expression of pro-apoptotic members of the same family). In addition, COX-2 inhibition is associated with modulation of p53 in several cell lines [29,30].

In EAC cells, Doxo+HMB reduced the expression of COX-2 in relation to controls (55%) and Doxo (53%). HMB modulated the arachidonic acid pathway, leading to a reduction in COX-2 and consequently, an increase in p53 and Bax expressions with a concomitant reduction in Bcl-xl expression, thereby triggering the intrinsic pathway of apoptosis. In the treatment with Doxo+HMB there was an intense process of cell death due to necrosis (note that in FIG. 2A the microscopy shows a much smaller number of cells per field) and thus the only detectable cells were in apoptosis because those in necrosis were removed by macrophages as this cell death process signals an inflammatory response

Considering the parameters for the diagnosis of cachexia (i.e. loss of body mass, alteration of food intake and increase in inflammatory parameters), it was found that the combination of Doxo+HMB, in relation to the Control and Doxo groups, was effective in preserving body mass, gastrocnemius muscle mass, and subcutaneous fat mass. The association Doxo+HMB also preserved the deposits of mesenteric fat in relation to Doxo and increased the food intake of the animals. It is noteworthy that food intake is an important component in weight loss related to cancer, especially because the lower protein synthesis is also related to the lower availability of nutrients [6].

The Doxo+HMB treatment was associated with modulation of inflammatory parameters in the serum and muscle of animals with EAC. Pro-inflammatory cytokines are related to tumor progression and the development of cachexia [31, 32]. Overexpression of IL-6 in skeletal muscle has been identified in patients with ovarian cancer, where it contributes to muscle atrophy by decreasing the half-life of proteins and increasing the activity of the 26S proteosome [33,34]. A recent study found that HMB decreased the expression of IL-6 in esophageal cancer cell lines [35]. In the present investigation treatments with Doxo alone, HMB alone, and Doxo+HMB all decreased the expression of IL-6 in the gastrocnemius muscle in relation to the Control group, with no differences between groups, demonstrating that HMB and Doxo reduced muscle IL-6 expression via the same mechanism.

Doxo+HMB reduced the content of IL-1β in the gastrocnemius in 47% and 43%, respectively, in relation to control and Doxo alone. The IL-1 pathway is also hyperactive in cancer patients and contributes in several ways to the development of cachexia. For example, it induces the synthesis of Activin A, which is related to skeletal muscle atrophy via NFκB and p38 MAPK, positively regulating E3 ligases that activate MURF-1 and Atrogin-1, both related to the inhibition of protein synthesis. Finally, increasing plasma concentration of tryptophan also increases the synthesis of serotonin in the hypothalamus, which in turn, contributes to the reduction of appetite [31,36]. The highest food intake observed in the Doxo+HMB group (FIG. 3A) is due to the lower levels of Il-1b.

In healthy animals, the serum levels of C-reactive protein were 0.07 mg/L, while in animals with TAE the levels obtained were 1.63 mg/L. In the animals treated with Doxo and HMB isolated, the values found were 2.39 mg/L and 0.31 mg/L, respectively, while in the group treated with the association, the lowest levels were verified, being 0.2 mg/L (FIG. 4D). The results obtained show that the animals in the control group had C-reative protein levels around 23 times above normal, indicating the occurrence of systemic inflammation related to tumor development. This result, associated with significant weight loss, reduced food intake, reduced muscle and fat mass, as well as elevated pro-inflammatory cytokines observed previously, reinforces the hypothesis of cachexia induction by inoculation of EAC cells in the mice peritoneum Balb/c.

Doxo treatment promoted an increase in C-reactive protein levels compared to controls (47%), while the Doxo+HMB treatment reduced this parameter by 92% in relation to Doxo alone. Systemic inflammation is related to greater mobilization of body reserves, reduced food intake, less response to cancer treatment and, consequently, worse prognosis.

The findings of the present study demonstrate that adding HMB to doxorubicin treatment has an anti-cachectic effect, as the combination increased food intake and preserved body mass. Adding HMB also reduced the expression of IL-1β in the gastrocnemius muscle and the serum levels of C-reactive protein, in relation to Doxo treatment alone, thereby protecting against the inflammation-driven catabolic potential related to this chemotherapy [37,38]. In a recent study with pigs submitted to LPS-induced muscle atrophy, the authors found that HMB supplementation promoted weight gain and improved food intake, and lowered serum IL-1β levels and muscle breakdown [39], further corroborating the results of the present study.

HMB modulated the arachidonic acid pathway, decreasing the expression of COX-2, inducing apoptosis mitochondrial pathway and increasing the expression of p53 and Bax while reducing the expression of Bcl-xl, in EAC cells. Therefore, this study found that HMB has an anti-cachectic activity in the setting of Doxo chemotherapy, as it increased food intake of animals with EAC, while reducing serum levels of C-reactive protein and the expression of IL-1β in the gastrocnemius muscle, in such a way contributing to the preservation of body reserves and to the protection against tumor cachexia.

The foregoing description and drawings comprise illustrative embodiments of the present inventions. The foregoing embodiments and the methods described herein may vary based on the ability, experience, and preference of those skilled in the art. Merely listing the steps of the method in a certain order does not constitute any limitation on the order of the steps of the method. The foregoing description and drawings merely explain and illustrate the invention, and the invention is not limited thereto, except insofar as the claims are so limited. Those skilled in the art who have the disclosure before them will be able to make modifications and variations therein without departing from the scope of the invention.

### REFERENCES

[1] Coussens L M, Web Z. Inflammation and cancer. Nature 2002; 19:860-7. https://doi.org/10.1038/nature01322.
[2] Murata M. Inflammation and cancer. Environ Health Prev Med. 2018; 23:2-8. https://doi.org/10.1186/s12199-018-0740-1.
[3] Soares J D P, Howell S L, Teixeira F J, Pimentel G D. Dietary amino acids and immunonutrition supplementation in cancer-induced skeletal muscle mass depletion: A mini review. Curr Pharm Des 2020; 26:1-8. https://doi.org/10.2174/1381612826666200218100420.
[4] Tisdale M J. Mechanisms of cancer cachexia. Physiol Rev 2009; 89:381-410. https://doi.org/10.1152/physrev.00016.2008.
[5] Loumaye A, Thissen J P. Biomarkers of cancer cachexia. Clin Biochem 2017:50; 1281-8. https://doi.org/10.1016/j.clinbiochem.2017.07.011.
[6] Baracos V E, Martin L, Korc M, Guttridge D C, Fearon K C H. Cancer-associated cachexia. Nat Rev Dis Primers 2018; 4:1-18. https://doi.org/10.1038/nrdp.2017.105
[7] Caillet P, Liluu E, Raynaud S A, Bonnefov M, Guerin O, Berrut G, et al. Association between cachexia, chemotherapy and outcomes in older cancer patients: a systematic review. Clin Nutr 2017; 36:1473-82. https://doi.org/10.1016/J.clnu.2016.12.003.
[8] Hulmi J J, Nissinen T A, Rasanen M, Degerman J, Lautaoja J H, Hemanthakumar K A, et al. Prevention of chemotherapy-induced cachexia by ACVR2B ligand blocking has different effects on heart and skeletal muscle. J Cachexia Sarcopenia Muscle 2018; 9:417-32. https://doi.org/10.1002/jcsm.12265.
[9] Biondo L A, Lima E A, Souza C O, Cruz M M, Cunha R D, Alonso-Vale M I, et al. Impact of doxorrubicin Treatment on the physiological functions of White adipose tissue. PLoS One 2016; 11:e0151548. https://doi.org/10.1371/journal.pone.0151548.
[10] Brain E G C, Mertens C, Girre V, Rousseau F, Blot E, Abadie S, et al. Impact of liposomal doxorubicin-based adjuvant chemotherapy on autonomy in women over 70 with hormone-receptor-negative breast carcinoma: A French Geriatric Oncology Group (GERICO) phase II multicenter trial. Crit Rev Oncol Hematol 2011; 80:160-70. https://10.1016/j.critrevonc.2010.10.003.
[11] Shahjehan F, Merchea A, Cochuyt J J, Li Z, Colibaseanu D T, Kasi P M. Body mass index and long-term outcomes in patients with colorectal cancer. Front Oncol 2018; 8:620. https://doi.org/10.3389/fonc.2018.00620.
[12] Sanz E A, Siles M G S, Dietitian L R F, Roldan R V R, Dominguez A R, Abiles J. Nutritional risk and malnutrition rates at diagnosis of cancer in patients treated in outpatient settings: Early intervention protocol. Nutrition 2019; 57:148-53. https://doi.org/10.1016/j.nut.2018.05.021.
[13] Van Koevering M, Nissen S. Oxidation of leucine and alpha-ketoisocaproate to beta-hydroxy-beta-methybutirate in vivo. Am J Physiol 1992; 262:E27-31. https://doi.org/10.1152/ajpendo.1992.262.1.E27.
[14] Molfino A, Gioia G, Rossi Fanelli F, Muscaritoli M. Beta-hydroxy-beta-methylbutyrate supplementation in healthy and disease: a systematic review of randomized trials. Amino Acids 2013; 45:1273-92. https://doi.org/10.1007/s00726-013-1592-z.
[15] Smith H J, Mukerji P, Tisdale M J. Attenuation of proteasoe-induced proteolysis in skeletal muscle by β-hydroxy-β-methylbutyrate in cancer-induced muscle loss. Cancer Res 2005; 65:277-83. https://doi.org/10.1007/s00726-013-1592-z.
[16] Caperuto E C, Tomatieli R V, Colquhoun A, Seelaender M C L, Rosa LFBPC. B-hydroxy-β-methylbutyrate supplementatin affects Walker 256 tumor-bearing rats in a time-dependent manner. Clinical Nutrition 2007; 26:117-22. https://doi.org/10.1016/j.clnu.2006.05.007.
[17] Nunes E A, Kuczera D, Brito G A P, Bonatto S J R, Yamazaki R K, Tanhoffer R A, Mund R C, Kryczyk M, Fernandes L C. β-hydroxy-βmethylbutyrate supplementation reduces tumor growth and tumor cell proliferation ex vivo and prevents cachexia in Walker 256 tumor-bearing rats by modifying nuclear factor-KB expression. Nutrition Research 2008; 28:487-93. https://doi.org/10.1016/J.nutres.2008.04.006.
[18] Kuczera D, Oliveira H H P, Guimaries F S F, Lima C, Alves L, Machado A F, Coelho I, Yamaguchi A, Donatti L, Naliwaiko K, Fernandes L C, Nunes E A. Bax/Bcl-2 protein expression ratio and leukocyte function are related to reduction of Walker-256 tumor growth after β-hydroxy-βmethylbutyrate (HMB) administration in wistar rats. Nutr Cancer 2012; 64:286-93. https://doi.org/10.1080/01635581.2012.647229.
[19] Yavas C, Yavas G, Acar H, Toy H, Yuce D, Akyrek S, et al. Amelioration of radiation-induced acute inflammation and mucosal atrophy by beta-hydroxy-beta-methylbutirate, L-glutamine and L-arginine: results of an experimental study. Support Care Cancer 2013; 21:883-8. https://doi.org/10.1007/s00520-012-1601-x.
[20] Yokota T, Hamauchi S, Yoshida Y, Yurijusa T, Suzuki M, Yamashita A, et al. A phase II study of HMB/Arg/Gln against oral mucositis induced by chemoradiotherapy for patients with head and neck cancer. Support Care Cancer 2018; 26:3241-8. https://doi.org/10.1007/s00520-018-4175-4.
[21] Reagan-Shaw S, Nihal M, Ahmad N. Dose translation from animal to human studies revisited. FASEB J 2008; 22:659-61. https://doi.org/10.1096/fj.07-9574LSF.
[22] Mota NSRS, Kviecinski M R, Zeferino R C, Oliveira D A, Bretanha L C, Ferreira S R S, et al. In vivo antitumor activity of byproducts of Passiflora edulis f. flavicarpa Deg. Rich in medium and long chain fatty acids evaluated through oxidative stress markers, cell cycle arrest and apoptosis induction. Food Chem Toxicol 2018; 118:557-65. http://doi.org/10.1016/J.fct.2018.06.010.
[23] Goel M K, Khanna P, Kishore J. Understanding survival analysis: Kaplan-Meier estimate. Int J Ayurveda Res 2010; 1:274-8. https://doi.org/10.4103/0974-7788.76794.
[24] Mcgahon A J, Martin S M, Bissonnette R P, Mahboubi A, Shi Y, Mogil R J, et al. The end of the (cell) line: methods for the study of apoptosis in vitro. Methods Cell Biol 1995; 46:153-85. https://doi.org/10.1016/s0091-679x(08)61929-9.
[25] Laemmli U K. Cleavage of structural protein during assembly of the head of bacteriophage T4. Nature 1970; 227:680-85. https://doi.org/10.1038/227680a0.
[26] Vasconcelos D A A, Giesbertz P, Souza D R, Vitzel K F, Abreu P, Marzuca-Nassr G N, et al. Oral L-glutamine pretreatment attenuates skeletal muscle atrophy induced by 24-h fasting in mice. J Nutr Biochem 2019; 70:202-14. https://doi.org/10.1016/j.jnutbio.2019.05.010.
[27] Bonetto A, Ruperrt J E, Barreto R, Zimmers T A. The colon-26 carcinoma tumor-bearing mouse as a model for the study of cancer cachexia. J Vis Exp 2016; 117: e54893. https://doi.org/10.3791/54893.
[28] Marzuca-Nassr G N, Murata G M, Martins A R, Vitzel K F, Crisma A R, Torres R P, et al. Balanced diet-fed fat-1 transgenic mice exhibit lower hindlimb suspension-induced soleus muscle atrophy. Nutrients 2017; 9:1-16. https://doi.org/10.3390/nu9101100.
[29] Sobolewski C, Cerella C, Dicato M, Ghibeli L, Diederich M. The role of cyclooxygenase-2 in cell proliferation and cell death in human malignancies. Int J Cell Biol 2010; 2010:215158. https://doi.org/10.1155/2010/215158.
[30] De Moraes E, Dar N A, De Moura Gallo C V, Hainaut P. Cross-talks between cyclooxygenase-2 and tumor suppressor protein p53: balancing life and death during inflammatory stress and carcinogenesis. Int J Cancer 2007; 121:929-37. https://doi.org/10.1002/ijc.22899.
[31] McDonald J J, McMillan D C, Laird B J A. Targeting IL-1α in cancer cachexia: a narrative review. Curr Opin Support Palliat Care 2018; 12:453-9. https://doi.org/10.1097/SPC.0000000000000398.
[32] Fearon K C H, Glass D J, Guttridge D C. Cancer cachexia: mediators, signaling and metabolic pathways. Cell Metab 2012; 16:153-66. https://doi.org/10.1016/j.cmet.2012.06.011.
[33] Dillon E L, Volpi E, Wolfe R R, Sinha S, Sanford A P, Arrastia C D, et al. Amino acid metabolism and inflammatory burden in ovarian cancer patients undergoing intense oncological therapy. Cllin Nutr Edinb Scotl 2007; 26:736-43. https://doi.org/10.1016/j.clnu.2007.07.004.
[34] Ebisui C, Tsujinaka T, Morimoto T, Kan K, Iijima S, Yano M, et al. Interleukin-6 induces proteolysis by activating intracellular proteases (cathepsins B and L, proteasome) in C2C12 myotubes. Cli Sci Lond Engl 1995; 89:431-9. https://doi.org/10.1042/cs0890431.
[35] Miyake S, Ogo A, Kubota H, Teramoto F, Hirai T. β-hydroxy-β-methylbutyrate suppresses NF-κB activation and IL-6 production in TE-1 cancer cells. In vivo 2019; 33:353-8. https://doi.org/10.21873/invivo.11481.
[36] Bent R, Moll L, Grabbe S, Bros M. Interleukin-1 beta—a friend or foe in malignancies? Int J Mol Sci 2018; 19:E2155. https://doi.org/10.3390/ijms19082155.
[37] Guigni B A, Callahan D M, Tourville T W, Miller M S, Fiske B, Voigt T, et al. Skeletal muscle atrophy and dysfunction in breast cancer patients: role for chemotherapy-derived oxidant stress. Am J Physiol Cell Physiol 2018; 315:744-56. https://doi.org/10.1152/ajpcell.00002.2018.
[38] Gorini S, De Angelis A, Berrino L, Malara N, Rosano G, Ferraro E. Chemotherapeutic drugs and mitochondrial dysfunction: focus on doxorubicin, trastuzumab and sunitinib. Oxid Med Cell Longev 2018; 18:7582730. https://doi.org/10.1155/2018/7582730.
[39] Duan Y, Zheng C, Zhong Y, Song B, Yan Z, Kong X, et al. Yin. β-hydroxy-β-methylbutyrate decreases muscle protein degradation via increased Akt/FoxO3a signaling and mitochondrial biogenesis in weanling piglets after lipopolysaccharide challenge. Food Funct 2019; 10:5152-65. https://doi.org/10.1039/c9fo00769e.

1. A method for inhibiting tumor growth in an animal undergoing chemotherapy treatment comprising administering to an animal in need thereof being treated with a chemotherapy agent from about 0.5 g to about 30 g of β-hydroxy-β-methylbutyrate (HMB).

2. The method of claim 1, wherein said HMB is selected from the group consisting of its free acid form, its salt, its ester and its lactone.

3. The method of claim 1, wherein said HMB is a calcium salt.

4. The method of claim 1, wherein HMB is in the free acid form.

5. The method of claim 1, wherein the chemotherapy agent is doxorubicin (Doxo).

6. A method for increasing survival of an animal undergoing chemotherapy treatment comprising administering to an animal in need thereof being treated with a chemotherapy agent from about 0.5 g to about 30 g of β-hydroxy-β-methylbutyrate (HMB).

7. The method of claim 6, wherein said HMB is selected from the group consisting of its free acid form, its salt, its ester and its lactone.

8. The method of claim 6, wherein said HMB is a calcium salt.

9. The method of claim 6, wherein HMB is in the free acid form.

10. The method of claim 6, wherein the chemotherapy agent is doxorubicin (Doxo).

11. A method for protecting against chemotherapy-induced weight loss in an animal undergoing chemotherapy treatment comprising administering to an animal in need thereof being treated with a chemotherapy agent from about 0.5 g to about 30 g of β-hydroxy-β-methylbutyrate (HMB).

12. The method of claim 11, wherein said HMB is selected from the group consisting of its free acid form, its salt, its ester and its lactone.

13. The method of claim 11, wherein said HMB is a calcium salt.

14. The method of claim 11, wherein HMB is in the free acid form.

15. The method of claim 11, wherein the chemotherapy agent is doxorubicin (Doxo).

16. A method for protecting against chemotherapy-induced inflammation in an animal undergoing chemotherapy treatment comprising administering to an animal in need thereof being treated with a chemotherapy agent from about 0.5 g to about 30 g of β-hydroxy-β-methylbutyrate (HMB).

17. The method of claim 16, wherein said HMB is selected from the group consisting of its free acid form, its salt, its ester and its lactone.

18. The method of claim 16, wherein said HMB is a calcium salt.

19. The method of claim 16, wherein HMB is in the free acid form.

20. The method of claim 16, wherein the chemotherapy agent is doxorubicin (Doxo).

21. A method for providing an anti-cachectic treatment in an animal undergoing chemotherapy treatment comprising administering to an animal in need thereof being treated with a chemotherapy agent from about 0.5 g to about 30 g of β-hydroxy-β-methylbutyrate (HMB).

22. The method of claim 21, wherein said HMB is selected from the group consisting of its free acid form, its salt, its ester and its lactone.

23. The method of claim 21, wherein said HMB is a calcium salt.

24. The method of claim 21, wherein HMB is in the free acid form.

25. The method of claim 21, wherein the chemotherapy agent is doxorubicin (Doxo).
